(19) United States
(12) Patent Application Publication
(10) Pub. No.: US 2011/0288004 A1
(43) Pub. Date: Nov. 24, 2011

(54) Viral Sequences and Uses Thereof

(75) Inventors: D. Grant McFadden, Gainsville, FL (US); Alexandra R. Lucas, Gainesville, FL (US); John W. Barrett, London (CA)

(73) Assignee: Robarts Research Institute

(21) Appl. No.: 12/993,191

(22) PCT Filed: Jun. 21, 2007
(86) PCT No.: PCT/US07/14593

§ 371 Date: Jan. 31, 2011

Related U.S. Application Data
(60) Provisional application No. 60/815,485, filed on Jun. 21, 2006.

(51) Int. Cl. A61K 38/16; C12N 15/63; C12N 5/10; C12P 21/00; C07K 14/065; A61P 29/00; A61P 11/00; A61P 27/14; A61P 17/00; A61P 11/06; A61P 9/00; A61P 9/10; A61P 25/00; A61P 3/10; A61P 13/12; A61P 35/02; A61P 25/28; A61P 19/02; A61P 7/06; A61P 35/00; C07H 21/00; C07K 19/00; A61K 31/7088; A61P 37/02; A61P 17/10; A61P 37/04; A61P 11/02; A61P 37/06; A61P 1/16; A61P 1/18; A61P 1/00; A61P 43/00; C12N 5/09

(52) U.S. Cl. 514/1.4; 435/375; 435/320.1; 435/325; 435/69.1; 530/350; 514/1.1; 514/21.2; 514/1.5; 514/20.8; 514/18.7; 514/1.7; 514/16.4; 514/1.9; 514/17.7; 514/6.9; 514/15.4; 514/19.6; 514/17.9; 514/16.8; 514/13.5; 514/16.6; 514/7.3; 514/19.2; 514/19.3; 514/19.4; 514/19.5; 536/23.72; 536/23.4; 514/44 R

(57) ABSTRACT

The invention features polypeptide and polynucleotide sequences based on the 134R sequence. In some embodiments, these sequences include a heterologous signal sequence, such as the myxoma virus T7 signal sequence. The invention also features methods for treating immunological disorders and neoplasms (e.g., cancer) using the polypeptides and nucleotides described herein. Finally, the invention features fusion proteins including the myxoma virus T7 signal sequence.

### BACKGROUND OF THE INVENTION

The invention relates to poxvirus polypeptides and polynucleotides and uses of such compounds in the treatment of diseases.

Tanapox virus (TPV) and Yaba Like Disease Virus (YLDV) are members of the genus Yatapoxvirus, which infects simians and other primates including humans. Nucleotide sequences of TPV and YLDV are over 98% identical and TPV and YLDV are serologically cross-reactive.

The YLDV 134R gene encodes a protein homologous to cellular proteins of the IL-10 family. This protein was described by Lee et al. (Virology 281:170-192, 2001) and Bartlett et al. (J. Gen. Virol. 85:1401-1412, 2004 The need exists for compositions for immune system modulation.

### SUMMARY OF THE INVENTION

In a first aspect, the invention features a substantially pure polypeptide including at least a fragment of TPV134R. The polypeptide may be biologically active (e.g., have a greater activity in a cell, as measured by STAT3 phosphorylation, as compared to human IL-10). The polypeptide may be expressed in a host cell. The polypeptide may be secreted from a mammalian host cell. At least a portion of the amino acid sequence (e.g., the entire amino acid sequence) of the polypeptide may be at least 60% (e.g., 70%, 80%, 90%, 95%, 98%, 99%, or even 100%) identical to amino acids 19-156 of TPV134R (SEQ ID NO:7). The polypeptide may further include a heterologous signal sequence (e.g., the amino acid sequence MDGRLVFLLASLAIVSDA (SEQ ID NO:12)). The signal sequence may be an N-terminal or C-terminal sequence. In some embodiments, the polypeptide includes or consists of the amino acid sequence of SEQ ID NO: 9 or 10. The invention also features a composition including any of the polypeptides described herein and a pharmaceutically acceptable carrier.

In another aspect, the invention features a substantially pure polypeptide including a TPV 134R protein, as defined herein.

In a related aspect, the invention features a method of treating a subject suffering from an immunomodulatory disorder including administering to the subject a 134R polypeptide, especially any of the peptides or pharmaceutical compositions described in the previous aspects.

In another aspect, the invention features a method of treating a subject suffering from a neoplasm including administering to the subject a substantially pure polypeptide including at least a fragment of TPV 134R.

In either of the two previous aspects, the polypeptide may be a polypeptide including or consisting of a sequence at least 60% (e.g., 70%, 80%, 90%, 95%, 98%, 99%, or even 100%) identical to amino acids 19-156 of TPV134R (SEQ ID NO:7) or a polypeptide including any other TPV134R fragment or variant described herein.

In another aspect, the invention features a method of inducing apoptosis in cell (e.g., a neoplastic cell, such as a cancer or tumor cell) including administering to the cell a substantially pure polypeptide including at least a fragment of TPV 134R.

The invention also features polynucleotides based on the TPV 134R sequence. In one aspect, the invention features a substantially pure polynucleotide encoding a biologically active polypeptide comprising a fragment of TPV 134R. In some embodiments, at least a portion of the codons have been altered to increase expression levels of the encoded protein when expressed in a mammalian cell (e.g., the GC₃ content of the polynucleotide may be greater than 30% or 50%). The codons may further be optimized for secretion from the mammalian cell. The polynucleotide may include a point mutation to remove a predicted splice site. The encoded polypeptide may further include a heterologous signal sequence (e.g., an N-terminal or C-terminal signal sequence such as MDGRLVFLLASLAIVSDA (SEQ ID NO:12)). In particular embodiments, the polynucleotide may include the sequence of Syn-134R (SEQ ID NO:4) or Syn2-134R (SEQ ID NO:6). The invention also features a vector and a cell (e.g., a mammalian cell, an insect cell, or any other cell described herein), each including any of the polynucleotides of the present aspect. The invention also features a pharmaceutical composition including a polynucleotide of the present aspect and a pharmaceutically acceptable carrier.

In another aspect, the invention features a method of producing a substantially pure polypeptide including culturing a cell (e.g., a mammalian cell an insect cell, or any other cell described herein) of the previous aspect under conditions that permit expression of the polypeptide encoded by the polynucleotide; and purifying the polypeptide, thereby producing an substantially pure polypeptide.

The invention also features a method of treating a subject suffering from an immunomodulatory disorder including administering to the subject any polynucleotide described in the previous aspects of the invention.

In another aspect, the invention features a method of treating a subject suffering from a neoplasm including administering to the subject any polynucleotide described in the previous aspects of the invention.

In another aspect, the invention features a method of inducing apoptosis in cell (e.g., a neoplastic cell, such as a cancer or tumor cell) by administering to the cell any of the polynucleotides described in a previous aspect of the invention.

In a final aspect, the invention features fusion protein including a myoxma T7 signal sequence and a heterologous sequence. The signal sequence may include MDGRLVFLLASLAIVSDA (SEQ ID NO: 12) or any variant thereof (e.g., those described herein). The invention also features polynucleotides encoding such fusion proteins.

In any of the treatment methods for immunomodulatory disorders described above, the disorder may be selected from the group consisting of acne vulgaris, acquired immune deficiency syndrome septic shock and other type of acute inflammation, acute respiratory distress syndrome, acute respiratory distress syndrome (ARDS), allergic intraocular inflammatory diseases, allergic rhinitis, ANCA-associated small-vessel vasculitis, inflammatory dermatoses, and Wegener's granulomatosis, ankylosing spondylitis, Addison's disease, arthritis, asthma, atherosclerosis, atopic dermatitis, atrophic gastritis, autoimmune complications of AIDS, autoimmune diseases, autoimmune hemolytic anemia, autoimmune hepatitis, Behcet's disease, Bell's palsy, bullous pemphigoid, celiac disease, cerebral ischaemia, chromic active hepatitis, chronic obstructive pulmonary disease, cirrhosis, CNS inflammatory disorder antigen-antibody complex mediated diseases, Cogan's syndrome, contact dermatitis, COPD, Crohn's disease, Cushing's syndrome, dermatitis, dermatomyositis, diabetes mellitus, discoid lupus erythematosus, encephalitis, eosinophilic fasciitis, erythema nodosum, exfoliative dermatitis, fibromyalgia, focal glomerulosclerosis, focal segmental glomerulosclerosis, food allergies, giant cell arteritis, glomerulonephritis, gout, gouty arthritis, graft-versus-host disease, granulomatorsis, Graves disease, habitual spontaneous abortions, hand eczema, Hashimoto's thyroiditis, Henoch-Schonlein purpura, hepatitis (e.g., viral hepatitis such as hepatitis A, herpes gestationis, hirsutism, idiopathic cerato-scleritis, idiopathic pulmonary fibrosis, idiopathic thrombocytopenic purpura, immune thrombocytopenic purpura inflammatory bowel or gastrointestinal disorders, inflammatory dermatoses, ischemic heart disease, leukemia, leukocyte adhesion deficiency, lichen planus, lipid histiocytosis, lupus nephritis, lymphomatous tracheobronchitis, macular edema, meningitis, multiple sclerosis, myasthenia gravis, myositis, necrotizing vasculitis, nonspecific fibrosing lung disease, osteoarthritis, pancreatitis, pemphigoid, pemphigoid gestationis, pemphigus, emphigus vulgaris, periodontitis, pernicious anemia, polyarteritis nodosa, polymyalgia rheumatica, polymyositis, post-dialysis syndrome, primary biliary cirrhosis, progressive systemic sclerosis, proliferative skin diseases, pruritis/inflammation, pruritus scroti, psoriasis, psoriatic arthritis, pulmonary histoplasmosis, Reiter's syndrome, relapsing polychondritis, Reynard's syndrome, rheumatic fever, rheumatoid arthritis, rosacea caused by sarcoidosis, rosacea caused by scleroderma, rosacea caused by Sweet's syndrome, rosacea caused by systemic lupus erythematosus, rosacea caused by urticaria, rosacea caused by zoster-associated pain, sarcoidosis, scleroderma, segmental glomerulosclerosis, septic shock syndrome, shoulder tendinitis or bursitis, Sjogren's syndrome, Still's disease, stroke-induced brain cell death, Sweet's disease, systemic lupus erythmatosus, systemic sclerosis, Takayasu's arteritis, temporal arteritis, thrombotic thrombocytopenic purpura, toxic epidermal necrolysis, transplant-rejection and transplant-rejection-related syndromes, tuberculosis, type 1 insulin-dependent diabetes mellitus, ulcerative colitis, uveitis, and vasculitis.

In any of the treatment methods for neoplams described above, the neoplasm may be a cancer selected from the group consisting of brain cancer, acute leukemia, acute lymphocytic leukemia, acute myelocytic leukemia, acute myeloblastic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendriglioma, schwannoma, meningioma, melanoma, neuroblastoma, and retinoblastoma, lung cancer, squamous cell carcinoma, adenocarcinoma, large cell carcinoma, and colon cancer.

By “134R protein” is meant a protein having substantial identity to the Tanapox 134R amino acid sequence (SEQ ID NO:7) or Yaba-Like Disease Virus (YLDV) 134R amino acid sequence (SEQ ID NO:8). Polypeptides of the invention substantially identical or homologous to TPV 134R polypeptides specifically exclude those consisting of the naturally occurring YLDV 134R sequence (SEQ ID NO:8) or a naturally occurring truncated form (amino acids 19-156 of YLDV 134R) described by Bartlett et al. (J. Gen. Virol. 85:1401-1412, 2004).

A nucleic acid molecule or polypeptide is said to be “substantially identical” to a reference molecule if it exhibits at least 50%, 55%, 60%, 65%, 68%, 75%, 85%, 90%, 95%, or 99% identity to the sequence of the reference molecule. For polypeptides, the length of comparison sequences is at least 10, 20, 25, 35, or 50 amino acids. For nucleic acid molecules, the length of comparison sequences is at least 30, 50, 60, 75, 100, or 150 nucleotides.

Alternatively, or additionally, two nucleic acid sequences are “substantially identical” if they hybridize under high stringency conditions. By “high stringency conditions” is meant conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (These are typical conditions for high stringency northern or Southern hybridizations.) High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually 16 nucleotides or longer for PCR or sequencing and 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

By “biologically active fragment” is meant a polypeptide fragment of a full length polypeptide that exhibits an activity (e.g., an immunomodulatory activity) that is at least 5%, 10%, 25%, 50%, 75%, 80%, 90%, or 95% of activity of the full length polypeptide. As used herein, the term “fragment” means at least 10, 30, 50, 60, 80, or 100 contiguous amino acids. Fragments of proteins described herein can be generated by methods known to those skilled in the art or may result from normal protein processing (e.g., removal of amino acids from the nascent polypeptide that are not required for biological activity or removal of amino acids by alternative mRNA splicing or alternative protein processing events).

A nucleotide which is “capable of expressing a polypeptide in a mammalian cell” means a nucleotide which, when operably linked to a promoter or other appropriate regulatory element and introduced into a mammalian cell cultured under conditions allowing for expression, will express the polypeptide encoded by the nucleotide at a level detectable using a method standard in the art (e.g., immunogenic techniques such as western blotting or ELISA or protein staining techniques such as silver staining). :Secreted” means that the expressed polypeptide is secreted from the host cell into the culture medium at a level detectable using a method standard in the art. “Capable of increased expression of a polypeptide in a mammalian cell” refers to a polynucleotide containing at least one (e.g., 2, 5, 10, 20) mutation(s) relative to a reference (e.g., wild-type) sequence which is expressed in a quantity at least 10%, 20%, 30%, 40%, 50%, 75%, 100%, 250%, 500%, 1000%, or 5000% greater than expression of the reference sequence under the same conditions.

“GC₃ content” refers to the proportion or percentage of nucleic acid codons within a coding sequence of a polynucleotide which have a guanine or cytosine at the third nucleic acid position of each codon.

By “splice site” is meant a nucleic acid sequence which causes an mRNA to be cleaved following transcription. The presence of a splice site within the coding sequence of an mRNA transcribed from a DNA molecule (for example, a vector) may decrease or result in no detectable expression of the encoded protein. A “predicted splice site” is a spliced site identified based on homology to known splice sites. Such sites can be identified using software known in the art including the software provided by Reese and Eeckman, Lawrence Berkeley National Laboratory, Genome Informatics Group or the software described in Hebsgaardet al., Nucleic Acids Res 1996, 24:3439-3452, 1996 and Brunak et al., J Mol Biol 220:49-65, 1991 (available from the Center for Biological Sequence Analysis at the Technical University of Denmark at http://www.cbs.dtu.dk/services/NetGene2/).

By a “substantially pure polypeptide” is meant a polypeptide (or a fragment or analog thereof) that has been separated from proteins and organic molecules that naturally accompany it. Typically, a polypeptide is substantially pure when it is at least 60%, by weight, free from the proteins and naturally-occurring organic molecules with which it is naturally associated. In other embodiments, the polypeptide is at least 75%, 80%, 90%, 95%, or 99%, by weight, pure. Purity can be measured by any appropriate method, e.g., by column chromatography, polyacrylamide gel electrophoresis, or HPLC analysis.

A polypeptide is substantially free of naturally associated components when it is separated from those proteins and organic molecules that accompany it in its natural state. Thus, a protein that is chemically synthesized or produced in a cellular system different from the cell in which it is naturally produced is substantially free from its naturally associated components. Accordingly, substantially pure polypeptides not only include those derived from eukaryotic organisms, but also those synthesized in expression systems, for example, E. coli or other prokaryotes, yeast, or insect cells.

“Mammalian cell” means a eukaryotic cell derived from a mammalian species. “Human mammalian cell” means a eukaryotic cell derived from humans. Examples of mammalian cells include Chinese Hamster Ovary (CHO) cells, PERC-6 cells, HeLA cells, COST cells, Hek293 cells, and other suitable cell types known to persons skilled in the art.

“Substantially pure nucleic acid molecule” means a nucleic acid molecule that is free of the components that naturally accompany it. For example, a substantially pure DNA is free of the genes that, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or that exists as a separate molecule (e.g., a cDNA or a genomic or cDNA fragment produced by PCR or restriction endonuclease digestion) independent of other sequences. It also includes a recombinant DNA that is part of a hybrid gene encoding additional polypeptide sequence.

By “vector” is meant a genetically engineered plasmid or virus, derived from, for example, a bacteriophage, adenovirus, retrovirus, poxvirus, herpesvirus, or artificial chromosome, that is used to transfer a polypeptide coding sequence, operably linked to a promoter, into a host cell, such that the encoded peptide or polypeptide is expressed within the host cell. A vector may be a gene therapy vector, i.e., a vector designed to transfer genetic material into the cells of a subject for a therapeutic benefit.

Vectors generally contain regulatory sequences, including promoters, operably linked to the polypeptide coding sequences. A “promoter” is a minimal nucleic acid sequence element sufficient to direct transcription. If desired, constructs of the invention can include promoter elements that are sufficient to render promoter-dependent gene expression controllable in a cell type-specific, tissue-specific, or temporal-specific manner, or inducible by external signals or agents. Such elements can be located in the 5′, 3′, or intron regions of a gene. Sequences are “operably linked” when a gene and one or more regulatory sequences are connected in such a way as to permit gene expression when the appropriate molecules (e.g., transcriptional activator proteins) are bound to the regulatory sequences.

“Immunomodulation,” “immunomodulatory,” or “immune modulation” refers to an alteration in the overall immunoreactivity of the immune system in a mammal, or alteration in the response of a cell, relative to an untreated control of the same type, upon treatment with an agent, such as a polypeptide or nucleic acid molecule of the present invention, or fragments and analogs thereof. Immunomodulation can be assayed using immune cells, for example, B cells, T cells, antigen-presenting cells, or any other cell that is involved in immune function. Immunomodulation can also be assayed by determining expression and/or activity of immune-related genes and proteins, or immune-related compounds, such as cytokines, cytokine receptors, or immunoglobulins.

“Immunosuppression” refers to a decrease in the overall immunoreactivity of the immune system upon administration of an immunomodulator in comparison to the immunoreactivity of an immune system that has not been contacted with the particular immunomodulator. “Immunostimulation” refers to a increase in the overall immunoreactivity of the immune system upon administration of an immunomodulator in comparison to the immunoreactivity of an immune system that has not been contacted with the particular immunomodulator. “Decreasing T cell stimulation” means lowering the level of T cell stimulation as measured by, for example, a chromium release assay. “Decreasing inflammation” means decreasing the number of inflammatory cells (leukocytes, for example eosinophils) in the target tissue by, preferably, two-fold. By “cell proliferation” is meant the growth or reproduction of similar cells. By “apoptosis” is meant the process of cell death where a dying cell displays a set of well-characterized biochemical hallmarks which include cytolemmal blebbing, cell soma shrinkage, chromatin condensation, and DNA laddering.

“Immunomodulator” refers to an agent that induces an immunomodulatory effect or alteration (e.g., immunosuppression, immunostimulation) as measured, for example, by an alteration of virulence in mutated viruses or a variety of immunoassays well known in the art (for example, chemotaxis assays as described herein).

An “anti-inflammatory” agent is an immunomodulatory agent capable of decreasing the overall inflammation or immune function upon administration to a subject.

Immunological or immunomodulatory disorders include, without limitation, acute inflammation, rheumatoid arthritis, transplant rejection, asthma, inflammatory bowel disease, uveitis, restenosis, multiple sclerosis, psoriasis, wound healing, lupus erythematosus, and any other autoimmune or inflammatory disorder that can be recognized by one of ordinary skill in the art. Subjects having immunological or immunomodulatory disorders may be treated by immunomodulation.

Examples of diseases and conditions related to inflammation include, acne vulgaris, acquired immune deficiency syndrome septic shock and other type of acute inflammation, acute respiratory distress syndrome, acute respiratory distress syndrome (ARDS), allergic intraocular inflammatory diseases, allergic rhinitis, ANCA-associated small-vessel vasculitis, inflammatory dermatoses, and Wegener's granulomatosis, ankylosing spondylitis, Addison's disease, arthritis, asthma, atherosclerosis, atopic dermatitis, atrophic gastritis, autoimmune complications of AIDS, autoimmune diseases, autoimmune hemolytic anemia, autoimmune hepatitis, Behcet's disease, Bell's palsy, bullous pemphigoid, celiac disease, cerebral ischaemia, chromic active hepatitis, chronic obstructive pulmonary disease, cirrhosis, CNS inflammatory disorder antigen-antibody complex mediated diseases, Cogan's syndrome, contact dermatitis, COPD, Crohn's disease, Cushing's syndrome, dermatitis, dermatomyositis, diabetes mellitus, discoid lupus erythematosus, encephalitis, eosinophilic fasciitis, erythema nodosum, exfoliative dermatitis, fibromyalgia, focal glomerulosclerosis, focal segmental glomerulosclerosis, food allergies, giant cell arteritis, glomerulonephritis, gout, gouty arthritis, graft-versus-host disease, granulomatorsis, Graves disease, habitual spontaneous abortions, hand eczema, Hashimoto's thyroiditis, Henoch-Schonlein purpura, hepatitis (e.g., viral hepatitis such as hepatitis A, herpes gestationis, hirsutism, idiopathic cerato-scleritis, idiopathic pulmonary fibrosis, idiopathic thrombocytopenic purpura, immune thrombocytopenic purpura inflammatory bowel or gastrointestinal disorders, inflammatory dermatoses, ischemic heart disease, leukemia, leukocyte adhesion deficiency, lichen planus, lipid histiocytosis, lupus nephritis, lymphomatous tracheobronchitis, macular edema, meningitis, multiple sclerosis, myasthenia gravis, myositis, necrotizing vasculitis, nonspecific fibrosing lung disease, osteoarthritis, pancreatitis, pemphigoid, pemphigoid gestationis, pemphigus, emphigus vulgaris, periodontitis, pernicious anemia, polyarteritis nodosa, polymyalgia rheumatica, polymyositis, post-dialysis syndrome, primary biliary cirrhosis, progressive systemic sclerosis, proliferative skin diseases, pruritis/inflammation, pruritus scroti, psoriasis, psoriatic arthritis, pulmonary histoplasmosis, Reiter's syndrome, relapsing polychondritis, Reynard's syndrome, rheumatic fever, rheumatoid arthritis, rosacea caused by sarcoidosis, rosacea caused by scleroderma, rosacea caused by Sweet's syndrome, rosacea caused by systemic lupus erythematosus, rosacea caused by urticaria, rosacea caused by zoster-associated pain, sarcoidosis, scleroderma, segmental glomerulosclerosis, septic shock syndrome, shoulder tendinitis or bursitis, Sjogren's syndrome, Still's disease, stroke-induced brain cell death, Sweet's disease, systemic lupus erythmatosus, systemic sclerosis, Takayasu's arteritis, temporal arteritis, thrombotic thrombocytopenic purpura, toxic epidermal necrolysis, transplant-rejection and transplant-rejection-related syndromes, tuberculosis, type 1 insulin-dependent diabetes mellitus, ulcerative colitis, and uveitis, vasculitis.

By “greater activity” is meant, under a given set of conditions, having either the ability to act either more rapidly (e.g., at least 10%, 25%, 50%, 100%, 250%, 500%, 1000%, 5000% more rapidly) or to a greater extent (e.g., at least 10%, 25%, 50%, 100%, 250%, 500%, 1000%, 5000% more) as compared to a reference (e.g., native or wild type 134R protein or human IL-10). For example, a polypeptide with greater activity in phosphorylating STAT3 than human IL-10 would generate either a greater amount of phosyphorylated STAT3 or generate phosphorylated STAT3 more rapidly than human IL-10, or both.

A “subject” may be a human or non-human animal (e.g., a mammal).

Other features and advantages of the invention will be apparent from the following Detailed Description, the drawings, and the claims.

### BRIEF DESCRIPTION OF THE DRAWINGS

FIG. 1A shows the Tanapox virus TPV 134R polynucleotide sequence (SEQ ID NO:1), the YLDV 134R polynucleotide sequence (SEQ ID NO:2), the Syn-134R polynucleotide sequence (with restriction sites underlined; SEQ ID NO:3), the Syn-134R polynucleotide coding sequence (SEQ ID NO:4), and the Syn2-134R polynucleotide sequence (SEQ ID NO:5).

FIG. 1B shows the Syn2-134R polynucleotide coding sequence (SEQ ID NO:6), the Tanapox virus 134R polypeptide sequence (SEQ ID NO:7), the YLDV 134R polypeptide sequence (SEQ ID NO:8), the Syn-134R polypeptide sequence (SEQ ID NO:9) and the Syn2-134R polypeptide sequence (SEQ ID NO:10).

FIG. 2 shows the myxoma virus M-T7 polypeptide sequence (SEQ ID NO:11) and the myxoma virus M-T7 polypeptide signal sequence (SEQ ID NO:12).

FIG. 3A is a photograph of a western blot showing detection of his/myc-tagged TPV134R expressed by recombinant baculovirus in SF-21 cells with anti-myc antibody. The wild-type 134R gene from Tanapox virus was cloned into a plasmid vector such that a myc/his tag was added to the C-terminus of the open reading frame. This tagged gene was then used to construct recombinant baculovirus that expresses the gene when used to infect insect (SF-21) cells. The myc-tagged product was associated with the infected cells (lane 2), but not the conditioned media (lane 1) implying that the tagged TPV-134 protein is not readily secreted.

FIG. 3B is a photograph of a western blot showing detection of hys/myc-tagged TPV134R and Syn-134R expressed in mammalian cells using an anti-myc antibody. In this figure, the Syn-134R constructs are referred to as Opti-134R clone 1 and Opti-134R clone 2. Clone 1 and 2 are identical, just independent isolates that were made during the cloning process. The wild-type 134R gene from Tanapox virus was cloned into a plasmid vector such that a myc/his tag was added to the C-terminus of the open reading frame. This construct did not express when transfected into either HEK293 or Cos7 mammalian cells (lanes 3). When the myc/his tagged version of the Syn-134R gene was used, it expressed well in both HEK293 and Cos7 mammalian cells (lanes 4 and 5).

FIG. 4 is a set of photographs of western blots showing transient expression of C-terminal myc/his-tagged 134R in Cos7 (Lane 1 to 6) and Hek293 cells (Lane 7 to 12). Forty-eight hours after transfection with either no plasmid (lanes 1 and 7), TPV134R-myc/his (lanes 2 and 8), or Syn-134R-myc/his (lanes 3-6 and 9-12), cells and media were collected, mixed with SDS gel loading buffer, separated on a 15% polyacrylamide gel, transferred to a nitrocellulose membrane, and the presence of myc-tagged fusion proteins were detected using a monoclonal anti-myc antibody. No expression was detected in the mock transfected cells (lane 1 and 7) or in the TPV134R-myc/his transfected cells (lane 2 and 8). Over expression of myc-tagged 134R protein was detected (arrow heads) in the Syn-134R-myc/his transfected cells (lanes 3-6 and 9-12).

FIGS. 5A and 5B are photographs of western blots showing expression and purification of myc/his-tagged 134R in baculovirus. FIG. 5A shows expression of a gene encoding 134R from TPV/YLDV in a recombinant baculovirus vector with a C-terminal myc/his tag. Both the cell lysates (lanes 1 and 2) and cell media (lanes 3 and 4) were tested for the presence of the tagged protein by an immunoblot probed with anti-myc monoclonal antibody. The expressed protein was not secreted and only associated with the cell lysates, not with the cell media at either 48 (lanes 1 and 3) or 72 hours (lanes 2 and 4) post-infection. FIG. 5B shows expression of a codon optimized gene (Syn-134R) encoding a 134R protein in a recombinant baculovirus vector with a C-terminal myc/his tag. Media was collected from baculovirus infected cells at 72 hours post-infection (lane 2). The myc/his-tagged 134R protein was purified by affinity chromatography using a HiTrap chelating column (Pharmacia) charged with nickel, according to the manufacturer's instructions. The eluted protein (lanes 5 and 6) reacted with the anti-myc monoclonal antibody by immunoblot.

FIG. 6 is a set of photographs of western blots showing that the myc/his-tagged 134R protein purified from baculovirus expressing Syn-134R was unexpectedly more active than endogenous human IL-10 in stimulating STAT3 phosphorylation in a human keratinocyte (HaCaT) cell-line. HaCaT cells were stimulated with human IL-10 or purified 134R protein for 20 min. The presence of phosphorylated STAT3 was detected using a monoclonal antibody that recognizes the phosphorylated form of STAT3. The total amount of STAT3 was determined on an identical immunoblot, using a separate monoclonal antibody that recognizes STAT3.

### DETAILED DESCRIPTION

The present invention features TPV 134R polypeptides, functional fragments thereof, and chimeric variants thereof (e.g., including a heterologous signal sequence), polynucleotides encoding such polypeptides, including sequences optimized for expression and/or secretion in mammalian cells, and methods of making such polypeptides and polynucleotides. We have further discovered that 134R proteins are more effective at phosphorylating STAT3 than human IL-10. Accordingly, the invention also features methods of treating immunoinflammatory disorders and neoplasms by administering the 134R polypeptides and/or polynucleotides to a subject in need of such treatment. Finally, the invention features fusion proteins with a myxoma T7 signal sequence.

### Tanapox 134R and Yaba Like Disease Virus 134R

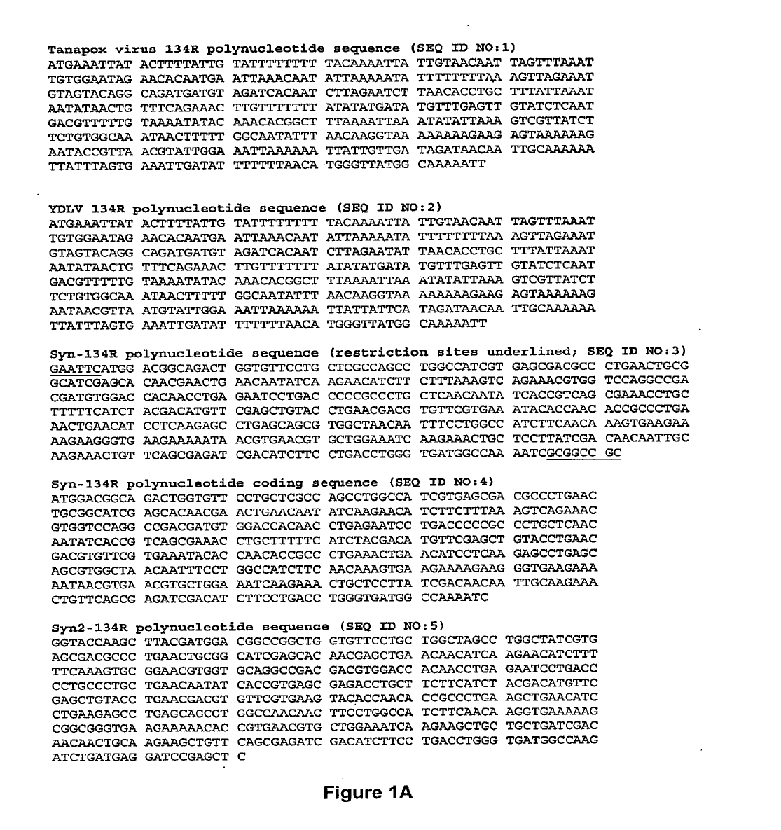

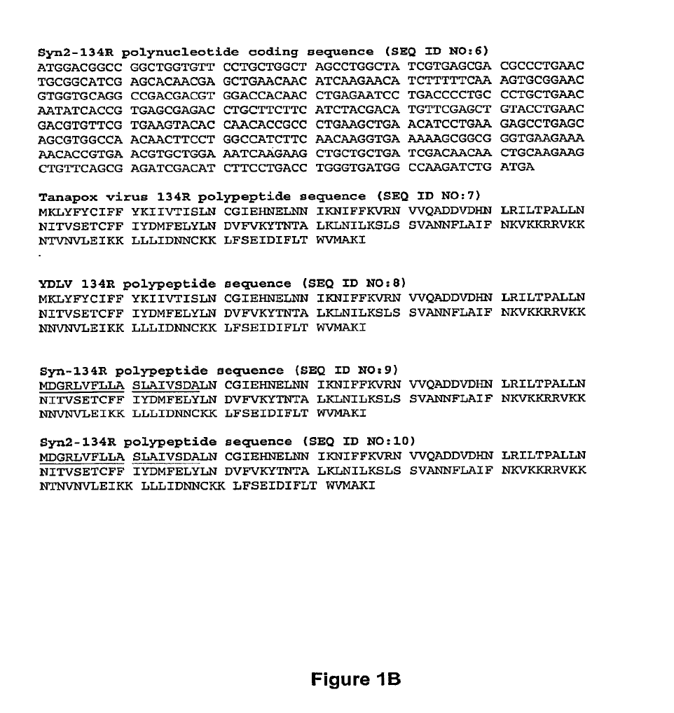

Tanapox 134R (TPV134R) (SEQ ID NOS:1 and 7; FIGS. 1A and 1B) and Yaba Like Disease Virus (YLDV134R) (SEQ ID NOS:2 and 8) are homologs of human IL-10 and are related to the IL-10 family of glycoproteins which include IL-10, IL-19, IL-20, IL-24, and IL-26. IL-10 is an immunoinhibitory protein which can exert immunoinhibition through phosphorylation of the STAT3 transcription factor. Previous work (Lee et al., Virology 281:170-192, 2001) has shown an alignment of YLDV134R (without the signal peptide) with human cytokines IL-24, IL-20 and IL-19. Of these, YLDV134R was most closely related to IL-24 with 28% amino acid identity and 55% similarity. More recently, Bartlett et al. (J Gen Virol 85:1401-1412, 2004) have performed bioinformatic analyses and protein modeling showing YLDV134R is predicted to be an α-helical glycoprotein most closely related to IL-24. Bartlett has further cloned and expressed YLDV134R. Expressing as C-terminal Flag-tagged protein in HEK293T cells, YLDV134R was found to be secreted as a monomeric glycoprotein.

IL-10 Family Members and their Receptors

Five IL-10 family members have been identified, IL-19, IL-20, IL-22, Il-24 (mda-7), and IL-26 (AK155). These cytokines mediate their activity through a number of receptors including IL-10R1, IL-10R2, IL-22R1, IL-20R2, and IL-20R1. IL-10, for example, binds and activates the IL-10R1 and IL10-R2 receptors. IL-24 appears to interact with the IL20R1, IL20R2, and IL22R1 receptors. Activation of these receptors has been shown to activate transcription factors STAT 1, 3, and 5 by phosphorlyation, thereby causing nuclear translocation of the protein, which then, in turn, activate their downstream genes. IL-10 is known to act as an inhibitor of inflammation and has been used in therapy for Crohn's disease.

### TPV134R is Most Closely Related to IL-24

As noted above, TPV134R bears the highest sequence similarity to IL-24 (also known as mda-7), one of the members of the IL-10 family of cytokines. IL-24 is a secreted glycoprotein. IL-24 has been identified as differentially expressed in melanomas, and studies have shown that IL-24 administration can result in growth suppression and apoptosis of tumor cells (Fisher et al., Cancer Biol Ther 2:(4:Suppl. 1) S23-S37, 2003) in a cancer-specific manner. Further studies have indicated that IL-24 can suppress growth of cancer cells from breast carcinoma (see, e.g., Bocangel et al., Cancer Gene Ther 13:958-968, 2006 and Su et al., Proc Natl Acad Sci USA 95:14400-14405, 1998), prostate carcinoma, lung cancer, glioblastoma, osteosacrcoma, fibrosacrcoma, colon carcinoma nasopharnygeal carcinoma, pancreatic carcinoma, cerical carcinoma, mesothelioma, ovarian cancer (see, e.g., PCT Publication No. WO03/087308), renal carcinoma, hepatocarcinoma, and neuroblastoma. IL-24 also inhibits angiogenesis and endothelial cell differentiation via the STAT3 signal transduction pathway following activation of the IL-22R (Ramesh et al., Cancer Res 63:5105-5113, 2003). IL-19 has also been indicated in the treatment of cancer such as ovarian cancer (see, e.g., U.S. Patent Application Publication No. 2003/0223958).

### Signal Sequence Replacement to Produce Syn-134R and Syn2-134R

We modified the YLDV134R and TPV134R sequences to replace the native signal sequence to improve expression of the mature protein. In general, polypeptides described herein (e.g., the 134R proteins) may be modified to incorporate any signal sequence known in the art, e.g., those described in the database described by Choo et al. (BMC Bioinformatics 2005, 6:249, 2005; available from the National University of Singapore at http://proline.bic.nus.edu.sg/spdb). In some embodiments, the protein is modified to incorporate a signal peptide that increases secretion of the protein from a cell.

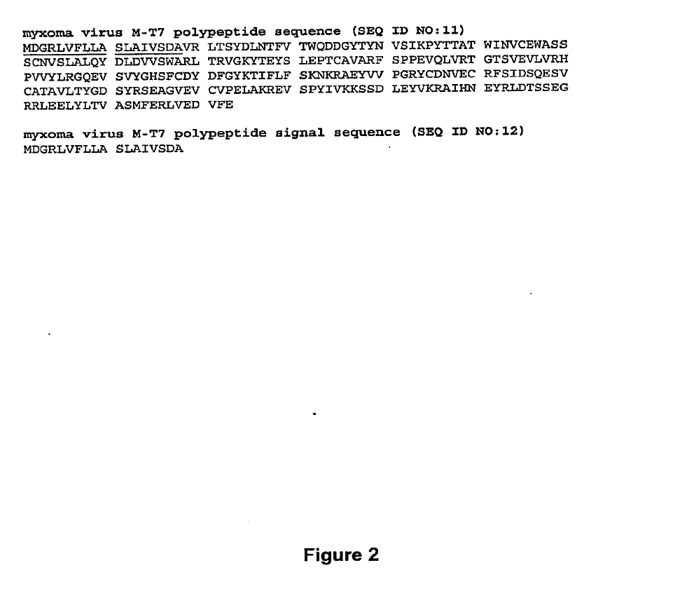

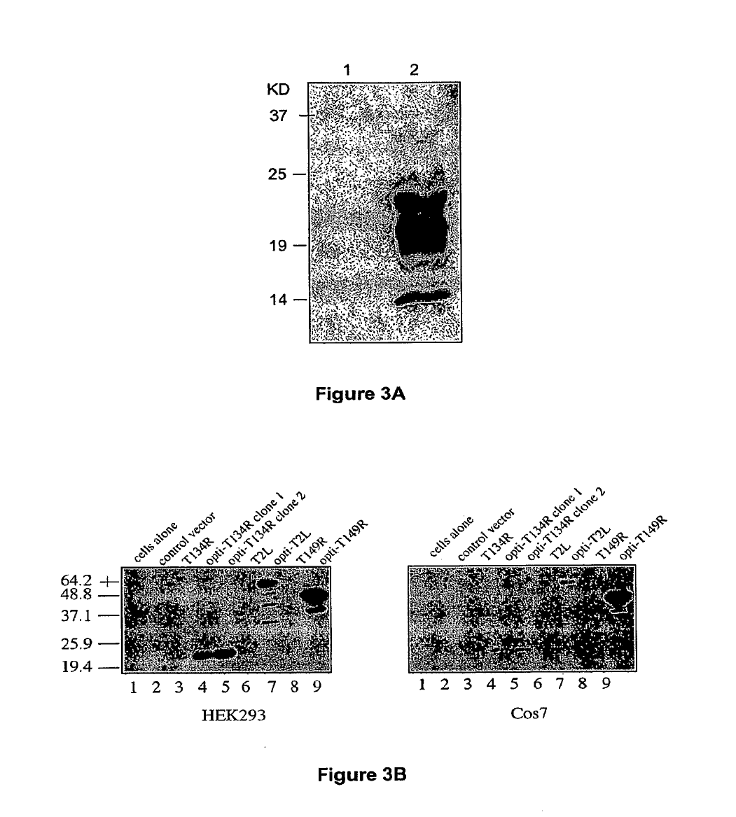

When the native TPV134R gene was cloned into a baculovirus expression system, it was expressed as a non-secreted protein (FIG. 3A). Analysis of the protein sequence using the SignalP program (available from the Center for Biological Sequence Analysis at the Technical University of Denmark at http://www.cbs.dtu.dk/services/signalP/), found that the N-terminal hydrophobic sequence had low probability of (i) being cleaved and (ii) functioning as a secretory signal sequence. Accordingly, the sequence coding for the 18 N-terminal amino acids of the 134R protein was replaced by sequence encoding the first 18 amino acids of the myxoma virus T7 sequence (MDGRLVFLLASLAIVSDA; SEQ ID NO:12; FIG. 2). Analysis of the myxoma virus T7 sequence (SEQ ID NO:11) using the SignalP software indicates that these first 18 amino acids are predicted to act as a signal sequence and are further predicted to have a cleavage site between amino acids 18 and 19. The nucleic acid sequences with the signal sequence replaced with the one from myxoma virus T7 are termed Syn-134R (SEQ ID NO: 4) and Syn2-134R (SEQ ID NO: 6), which correspond to Yaba Like Disease Virus (YLDV134R) (SEQ ID NOS:2 and 8) and Tanapox 134R (TPV134R) (SEQ ID NOS:1 and 7; FIGS. 1A and 1B), respectively

Syn-134R myc/his Tagged Protein is More Active than Human IL-10

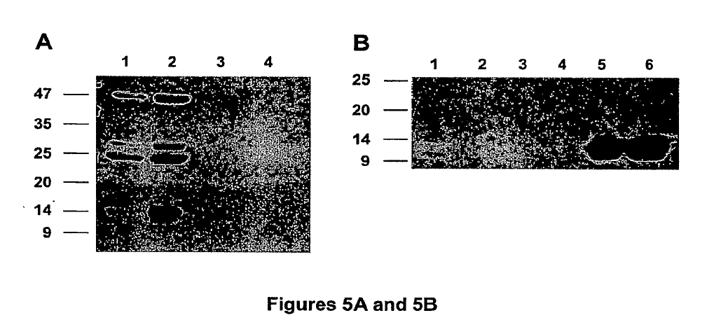

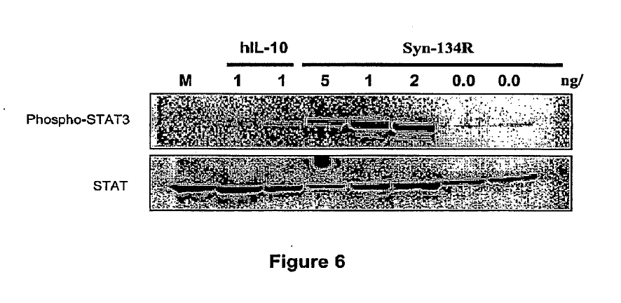

When members of the IL-10 family of cytokines are used to treat the human keratinocytic cell line HaCaT, they induce phosphorylation of the intracellular messenger protein STAT3. When the myc/his-tagged Syn-134R, purified from a recombinant baculovirus expression system (FIGS. 5A and 5B), was incubated with HaCaT cells for 20 minutes, the induction of phosphorylated STAT3 was observed by an immunoblot of the cell lysates probed with an antibody specific to phosphorylated STAT3. An identical immunoblot probed with a second antibody that detects all forms of STAT3 was used to indicate the total amounts of STAT3 protein in each sample (FIG. 6). From these experiments, Syn-134R was observed to induce phosphorylation of STAT3 in a dose-dependent fashion. Surprisingly, it was more active than commercially available human IL-10. As administration of IL-10 reduces inflammation, and IL-24 administration results in selective apoptosis of cancer cells, 134R and the fragments and variants described herein can thus be used to treat any condition or disease where increasing the amount of IL-10 family members is desirable. These conditions include immunomodulatory disorders and neoplasms (e.g., any of those described herein).

### Protein Expression

In general, polypeptides of the invention (e.g., Syn-134R) or for use in the methods of the invention may be produced by transformation or transfection of a suitable host cell with all or part of a cDNA fragment encoding the polypeptide in a suitable expression vehicle.

Those skilled in the field of molecular biology will understand that any of a wide variety of expression systems may be used to provide the recombinant protein. The precise host cell used is not critical to the invention. The polypeptide may be produced in a prokaryotic host (e.g., E. coli) or in a eukaryotic host (yeast cells, e.g., Saccharomyces cerevisiae, insect cells, e.g., Sf21 cells, or mammalian cells, e.g., COS 1, COS7, HEK293, NIH 3T3, or HeLa cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual, P. H. Pouwels et al., 1985, Supp. 1987).

One preferred expression system is the baculovirus system (using, for example, the vector pBacPAK9 or the vectors described herein) available from Clontech (Pal Alto, Calif.). If desired, this system may be used in conjunction with other protein expression techniques, for example, the myc-tag approach described by Evan et al. (Mol Cell Biol 5:3610-3616, 1985).

Alternatively, a protein is produced by a stably-transfected mammalian cell line. A number of vectors suitable for stable transfection of mammalian cells are available to the public, e.g., see Pouwels et al. (supra); methods for constructing such cell lines are also publicly available, e.g., in Ausubel et al. (supra). In one example, cDNA encoding the protein is cloned into an expression vector which includes the dihydrofolate reductase (DHFR) gene. Integration of the plasmid and, therefore, the protein-encoding gene into the host cell chromosome is selected for by inclusion of 0.01-300 mM methotrexate in the cell culture medium (as described in Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al. (supra); such methods generally involve extended culture in medium containing gradually increasing levels of methotrexate. DHFR-containing expression vectors commonly used for this purpose include pCVSEII-DHFR and pAdD26SV(A) (described in Ausubel et al., supra). Any of the host cells described above or, preferably, a DHFR-deficient CHO cell line (e.g., CHO DHFR⁻ cells, ATCC Accession No. CRL 9096) are among the host cells preferred for DHFR selection of a stably-transfected cell line or DHFR-mediated gene amplification.

Once the recombinant protein is expressed, it is purified or isolated, e.g., using affinity chromatography. In one example, an antibody (e.g., produced as described herein) is attached to a column and used to isolate the protein. Lysis and fractionation of protein-harboring cells prior to affinity chromatography are performed by standard methods (see, e.g., Ausubel et al., supra). In another example, proteins are purified or substantially purified from a mixture of compounds such as an extract or supernatant obtained from cells (Ausubel et al., supra). Standard purification techniques can be used to progressively eliminate undesirable compounds from the mixture until a single compound or minimal number of effective compounds has been isolated.

Once isolated, the recombinant protein can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides of the invention, particularly short protein fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful protein fragments or analogs (described herein).

Polypeptides can be attached to any one of a variety of tags. Tags can be amino acid tags or chemical tags and can be added for the purpose of purification (for example, a 6-histidine tag for purification over a nickel column or a myc tag). Various labels can be used as means for detecting binding of a protein to a second protein, for example, to a chemokine or a chemokine receptor. Polypeptides can also be linked to toxins. Proteins linked to toxins can be used, for example, to target toxic drugs to malignant tumors if the protein has the ability localize to the tumor.

### Therapeutic Methods Employing Polypeptides and Pharmaceutical Compositions

Based on the development of 134R sequences and variants described herein, the invention features methods of treating or preventing conditions and disorders including immunological disorders and neoplasms by administering the 134R polypeptide sequences disclosed herein to a patient (e.g., a patient in need of such treatment).

Polypeptides described herein (or fragments or analogs thereof) that exhibit anti-cytokine activity, anti-inflammatory activity, anti-neoplastic activity (e.g., apoptotic activity) and exhibit a decrease in leukocyte chemotaxis activity are considered particularly useful in the invention; such polypeptides may be used, for example, as therapeutics to decrease the immunoreactivity in a subject with rheumatoid arthritis or any disorder described herein. Other immunological disorders that may be treated using an immunosuppressive agent, or an agent that reduces the immune function, include acute inflammation, allergic reactions, asthmatic reactions, inflammatory bowel diseases (i.e., Crohn's disease and ulcerative colitis), transplant rejection, and restenosis, or any of the immunomodulatory conditions described herein. In other embodiments, a polypeptide that enhances or induces apoptosis (e.g., of a cancer cell) may be used in the treatment of a neoplasm such as a cancer (e.g., brain cancer, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendriglioma, schwannoma, meningioma, melanoma, neuroblastoma, and retinoblastoma, lung cancer, squamous cell carcinoma, adenocarcinoma, large cell carcinoma, and colon cancer).

Treatment or prevention of diseases resulting from an immunomodulatory disorder or neoplasm is accomplished, for example, by modulating the function of a immunoregulatory protein by delivering a polypeptide described herein to the appropriate cells. It is also possible to correct an immune defect by modifying the physiological pathway (e.g., a signal transduction pathway), in which the immunoregulatory protein participates, by delivering a polypeptide or nucleic acid molecule to the appropriate cells.

Direct administration of a recombinant polypeptide described herein, or nucleic acid molecule, either to the site of a potential or actual disease-affected tissue (for example, by injection), or systemically for treatment of, for example, an autoimmune or inflammatory disorder or a neoplasm, can be performed accordingly to any conventional recombinant protein administration technique known in the art or described herein. The actual dosage depends on a number of factors known to those of ordinary skill in the art, including the size and health of the individual subject, but generally, between 0:1 mg and 100 mg inclusive are administered per day to an adult in any pharmaceutically-acceptable formulation.

A polypeptide described herein may be mixed and administered with a pharmaceutically-acceptable diluent, carrier, or excipient, at a pharmaceutically effective dose. Conventional pharmaceutical practice may be employed to provide suitable formulations or compositions to administer polypeptides described herein to subjects suffering from an immunomodulatory disorder. Any appropriate route of administration may be employed, for example, parenteral, intravenous, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in art for making formulations are found in, for example, Remington's The Science and Practice of Pharmacy, Lippincott Williams & Wilkins, 21^{st} ed., 2005. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for polypeptides include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

### Myxoma T7 Signal Sequence

In another aspect, the invention features a fusion polypeptide including a myxoma T7 signal sequence (e.g., amino acids 1-18 of the myxoma virus T7 protein (SEQ ID NO:11)) (FIG. 2) and a heterologous sequence. Desirably, the addition of the T7 sequence increases secretion of the heterologous sequence from the cell (e.g., from any cell described herein or known in the art, such as a mammalian cell) in which it is produced, as compared to in the absence of the sequence. In addition, any functional fragment of the signal sequence can be used (e.g., 1, 2, 3, 4, 5, 6, 7, 8, 9, 10, 11, 12, 13, 14, or 15 amino acids may be deleted from either the N-terminal end or the C-terminal end of the signal sequence shown in FIG. 2. In other embodiments, point mutation, insertions, or deletions may be present in the signal sequence. Such changes can be introduced using molecular biological techniques well known in the art. Any appropriate heterologous protein (e.g., any protein known the art) can be chosen to be used in conjunction with the myxoma T7 sequence.

### Polynucleotides Coding for TPV134R

The invention also features polynucleotides encoding TPV134R and variants of TPV134R described herein. In particular, the invention features polynucleotides which include sequences encoding biologically active fragments of the TPV134R, splice-site and codon optimized versions of TPV134R, and chimeric TPV134R sequences (e.g., polynucleotides including sequence coding for the mature 19-156 amino acids of TPV134R with a heterologous signal sequence). Such polynucleotides can be generated using molecular biological techniques known in the art.

While it is often possible to express poxvirus proteins in a baculovirus expression system, it is desirable to express such proteins in a mammalian cell. Transient expression of viral genes from certain poxviruses in uninfected mammalian cells can be unexpectedly inefficient. The reasons for poor expression levels can be due to suboptimal codon usage, cryptic splice sites, polymerase II termination sequences, or motifs that lead to mRNA instability.

Expression of poxvirus sequences described herein in mammalian cells was achieved by introducing two general sets of changes into the Tanapox and Yaba Like Disease Virus (YLDV) 134R sequences. The first set of changes was designed to remove predicted splice sites. The second set of changes introduced silent mutations to introduce codons favorable for mammalian expression. The changes described herein allow for high levels of protein expression from sequences which are otherwise unable to express or express poorly in mammalian systems. Transient expression of individual viral proteins allows for the study of the specific gene products without the complications of the background contributions from the other viral proteins.

Splice Site Removal

Transcripts not normally expressed the nucleus of mammalian cells may contain splice sites which render the sequence unable to expressed efficiently. Processing sequences through software that searches for cryptic splice sites using software available from the Berkeley Drosophila Genome Project (University of California, Berkeley) http://www.friutfly.org/cgi-bin/seq_tools/splice.pl can be used to predict potential splice sites. Other software known in the art (e.g., software described herein) may likewise be used to identify potential or putative splice sites. Such changes were introduced into the 134R polynucleotide sequences (SEQ ID NO's:4 and 6) to generate the Syn-134R and Syn2-134R, respectively.

Codon Optimization

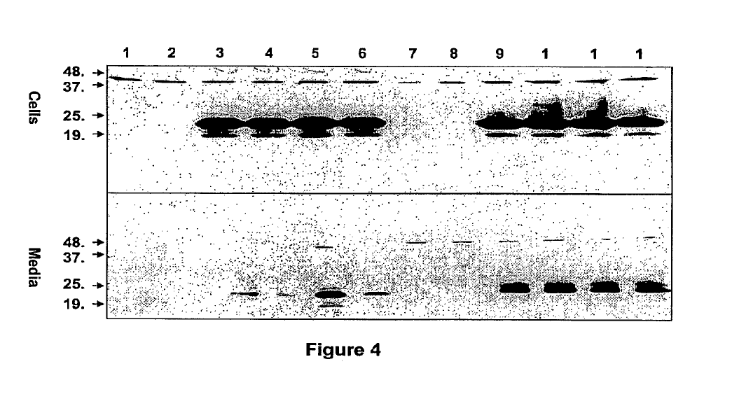

A second set of silent mutations can be introduced into the 134R sequence to generate codon optimized versions of the polynucleotides coding for these genes. As poxvirus genes are normally transcribed in the cytoplasm and never encounter the nucleus, there has not been any selection pressure exerted by host cell nucleus-resident pathways: To overcome these expression problems, changes were introduced into the 134R gene. In one example, the percent GC₃ in the 134R gene was increased from under 14% to over 75%. Here, no expression was detected in the mock transfected cells (FIG. 4, lanes 1 and 7), or in the TPV134R transfected cells (FIG. 4, lane 2 and 8). Overexpression of myc-tagged 134R protein was detected in the Syn-134R transfected cells (FIG. 4, lanes 3-6 and 9-12).

### Gene Therapy

A polynucleotide described herein or fragment thereof encoding an immunomodulatory polypeptide can be used in immunomodulatory or anti-neoplastic gene therapy. For example, to increase apoptosis of a tumor a functional, codon optimized 134R gene (e.g., the sequences described herein) or fragment thereof may be introduced into cells at the site of a tumor. Polynucleotides codon optimized for mammalian expression are especially useful in the treatment methods of the invention.

Gene transfer is achieved using viral vectors, as well as by non-viral means. Transplantation of genes into the affected tissues of a subject can also be accomplished by transferring a codon optimized gene or fragment thereof described herein into a cultivatable cell type ex vivo, after which the cell (or its descendants) is injected into a targeted tissue.

Retroviral vectors, adenoviral vectors, adeno-associated viral vectors, or other viral vectors with the appropriate tropism for cells may be used as a gene transfer delivery system for a therapeutic gene construct containing a codon optimized gene. Numerous vectors useful for this purpose are known (see, for example, Miller, Human Gene Ther 15-14, 1990; Friedman, Science 244:1275-1281, 1989; Eglitis and Anderson, BioTechniques 6:608-614, 1988; Tolstoshev and Anderson, Curr Opin Biotechnol 1:55-61, 1990; Sharp, Lancet 337:1277-1278, 1991; Cornetta et al., Nucleic Acid Res Mol Biol 36:311-322, 1987; Anderson, Science 226:401-409, 1984; Moen, Blood Cells 17:407-416, 1991; and Miller and Rosman, Biotechniques 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N Engl J Med 323:370, 1990; Anderson et al., U.S. Pat. No. 5,399,346).

Non-viral approaches, for the introduction of therapeutic DNA into cells, include transfection in vitro, by means of any standard technique, including but not limited to, calcium phosphate, DEAE dextran, electroporation, protoplast fusion, and liposomes. For example, a codon optimized gene described herein may be introduced into a tumor cell by lipofection (Feigner et al., Proc Natl Acad Sci USA 84:7413, 1987; Ono et al., Neuroscience Lett 117:259, 1990; Brigham et al., Am J Med Sci 298:278, 1989; Staubinger and Papahadjopoulos, Methods Enzymol 101:512, 1983); asialorosonucoid-polylysine conjugation (Wu and Wu, J Biol Chem 263:14621, 1988; Wu et al., J Biol Chem 264:16985, 1989); or microinjection under surgical conditions (Wolff et al., Science 247:1465, 1990).

For any of the above approaches, the therapeutic construct containing the codon optimized gene is preferably applied to the site of the malignancy or inflammation and cytotoxic damage (for example, by injection), but may also be applied to tissue in the vicinity of the malignancy or inflammation and cytotoxic damage, or even to a blood vessel supplying these areas.

In the gene therapy constructs, expression of the codon optimized gene is directed from any suitable promoter (e.g., the human cytomegalovirus, simian virus 40, or metallothionein promoters), and its production is regulated by any desired mammalian regulatory element. For example, if desired, enhancers known to direct preferential gene expression in endothelial or epithelial cells may be used to direct protein expression of the codon optimized gene. Such enhancers include, without limitation, the lung specific promoters (e.g., surfactant), and gut specific regulatory sequences.

Gene therapy is also accomplished by direct administration of a codon optimized mRNA to a tumor. This mRNA may be produced and isolated by any standard technique, but is most readily produced by in vitro transcription codon optimized cDNA under the control of a high efficiency promoter (e.g., the T7 promoter). Administration of the mRNA to malignant cells is carried out by any of the methods for direct nucleic acid administration described above.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth, and follows in the scope of the appended claims.

All publications, patents, and patent applications mentioned in this specification, including U.S. Provisional Application No. 60/815,485, filed Jun. 21, 2006, are incorporated by reference to the same extent as if each independent publication, patent, or patent application was specifically and individually indicated to be incorporated by reference.

What is claimed is:

1. A substantially pure polypeptide comprising at least a fragment of TPV134R.

2. The polypeptide of claim 1, wherein said polypeptide is biologically active.

3. The polypeptide of claim 2, wherein said polypeptide has greater activity in a cell, as measured by STATS phosphorylation, as compared to human IL-10.

4. The polypeptide of claim 1, wherein at least a portion of the amino acid sequence of said polypeptide is at least 90% identical to amino acids 19-156 of TPV134R (SEQ ID NO:7).

5. The polypeptide of claim 4, wherein said amino acid sequence is at least 90% identical to amino acids 19-156 of TPV134R (SEQ ID NO:7).

6. The polypeptide of claim 4, wherein said amino sequence is amino acids 19-156 of TPV134R (SEQ ID NO:7).

7. The polypeptide of claim 1, further comprising a heterologous signal sequence.

8. The polypeptide of claim 7, wherein said signal sequence is an N-terminal sequence.

9. The polypeptide of claim 7, wherein said signal sequence comprises the amino acid sequence MDGRLVFLLASLAIVSDA (SEQ ID NO: 12).

10. The polypeptide of claim 1 comprising the amino acid sequence of SEQ ID NO:9 or 10.

11. The polypeptide of claim 1 consisting of the amino acid sequence of SEQ ID NO:9 or 10.

12. A substantially pure polypeptide comprising a TPV134R protein.

13. A pharmaceutical composition comprising:
(a) a polypeptide comprising a biologically active fragment of TPV134R; and
(b) a pharmaceutically acceptable carrier.

14. A method of treating a subject suffering from an immunomodulatory disorder comprising administering to said subject a substantially pure polypeptide comprising at least a fragment of a 134R protein.

15. The method of claim 14, wherein said polypeptide comprises a sequence at least 90% identical to amino acids 19-156 of TPV134R (SEQ ID NO:7).

16. The method of claim 15, wherein said polypeptide comprises a sequence identical to 19-156 of TPV134R (SEQ ID NO:7).

17. The method of claim 16, wherein said polypeptide consists of a sequence identical to 19-156 of TPV134R (SEQ ID NO:7).

18. The method of claim 14, wherein said immunomodulatory disorder is selected from the group consisting of acne vulgaris, acquired immune deficiency syndrome, septic shock and other type of acute inflammation, acute respiratory distress syndrome, acute respiratory distress syndrome (ARDS), allergic intraocular inflammatory diseases, allergic rhinitis, ANCA-associated small-vessel vasculitis, inflammatory dermatoses, and Wegener's granulomatosis, ankylosing spondylitis, Addison's disease, arthritis, asthma, atherosclerosis, atopic dermatitis, atrophic gastritis, autoimmune complications of AIDS, autoimmune diseases, autoimmune hemolytic anemia, autoimmune hepatitis, Behcet's disease, Bell's palsy, bullous pemphigoid, celiac disease, cerebral ischaemia, chromic active hepatitis, chronic obstructive pulmonary disease, cirrhosis, CNS inflammatory disorder, antigen-antibody complex mediated diseases, Cogan's syndrome, contact dermatitis, COPD, Crohn's disease, Cushing's syndrome, dermatitis, dermatomyositis, diabetes mellitus, discoid lupus erythematosus, encephalitis, eosinophilic fasciitis, erythema nodosum, exfoliative dermatitis, fibromyalgia, focal glomerulosclerosis, focal segmental glomerulosclerosis, food allergies, giant cell arteritis, glomerulonephritis, gout, gouty arthritis, graft-versus-host disease, granulomatosis, Graves disease, habitual spontaneous abortions, hand eczema, Hashimoto's thyroiditis, Henoch-Schonlein purpura, hepatitis (e.g., viral hepatitis such as hepatitis A, herpes gestationis, hirsutism, idiopathic cerato-scleritis, idiopathic pulmonary fibrosis, idiopathic thrombocytopenic purpura, immune thrombocytopenic purpura, inflammatory bowel or gastrointestinal disorders, inflammatory dermatoses, ischemic heart disease, leukemia, leukocyte adhesion deficiency, lichen planus, lipid histiocytosis, lupus nephritis, lymphomatous tracheobronchitis, macular edema, meningitis, multiple sclerosis, myasthenia gravis, myositis, necrotizing vasculitis, nonspecific fibrosing lung disease, osteoarthritis, pancreatitis, pemphigoid, pemphigoid gestationis, pemphigus, emphigus vulgaris, periodontitis, pernicious anemia, polyarteritis nodosa, polymyalgia rheumatica, polymyositis, post-dialysis syndrome, primary biliary cirrhosis, progressive systemic sclerosis, proliferative skin diseases, pruritis/inflammation, pruritus scroti, psoriasis, psoriatic arthritis, pulmonary histoplasmosis, Reiter's syndrome, relapsing polychondritis, Reynard's syndrome, rheumatic fever, rheumatoid arthritis, rosacea caused by sarcoidosis, rosacea caused by scleroderma, rosacea caused by Sweet's syndrome, rosacea caused by systemic lupus erythematosus, rosacea caused by urticaria, rosacea caused by zoster-associated pain, sarcoidosis, scleroderma, segmental glomerulosclerosis, septic shock syndrome, shoulder tendinitis or bursitis, Sjogren's syndrome, Still's disease, stroke-induced brain cell death, Sweet's disease, systemic lupus erythmatosus, systemic sclerosis, Takayasu's arteritis, temporal arteritis, thrombotic thrombocytopenic purpura, toxic epidermal necrolysis, transplant-rejection and transplant-rejection-related syndromes, tuberculosis, type 1 insulin-dependent diabetes mellitus, ulcerative colitis, uveitis, and vasculitis.

19. A method of treating a subject suffering from a neoplasm comprising administering to said subject a substantially pure polypeptide comprising at least a fragment of a 134R protein.

20. The method of claim 19, wherein said polypeptide comprises a sequence at least 90% identical to amino acids 19-156 of TPV134R (SEQ ID NO:7).

21. The method of claim 20, wherein said polypeptide comprises a sequence identical to 19-156 of TPV134R (SEQ ID NO:7).

22. The method of claim 21, wherein said polypeptide consists of a sequence identical to 19-156 of TPV134R (SEQ ID NO:7).

23. The method of claim 19, wherein said neoplasm is a cancer selected from the group consisting of brain cancer, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, Hodgkin's disease, non-Hodgkin's disease, Waldenstrom's macroglobulinemia, heavy chain disease, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendriglioma, schwannoma, meningioma, melanoma, neuroblastoma, and retinoblastoma, lung cancer, squamous cell carcinoma, adenocarcinoma, large cell carcinoma, and colon cancer.

24. A method of inducing apoptosis in cell comprising administering to said cell a substantially pure polypeptide comprising at least a fragment of a 134R protein.

25. The method of claim 24, wherein said cell is a neoplastic cell.

26. A substantially pure polynucleotide encoding a biologically active polypeptide comprising a fragment of a 134R protein.

27. The polynucleotide of claim 26, wherein at least a portion of the codons have been altered to increase expression levels of the encoded protein when expressed in a mammalian cell.

28. The polynucleotide of claim 27, wherein the GC₃ content of said polynucleotide is greater than 30%.

29. The polynucleotide of claim 27, wherein the GC₃ content of said polynucleotide is greater than 50%.

30. The polynucleotide of claim 27, wherein said polynucleotide comprises a point mutation to remove a predicted splice site.

31. The polynucleotide of claim 26, wherein said polypeptide further comprises a signal sequence.

32. The polynucleotide of claim 31, wherein said signal sequence is an N-terminal signal sequence.

33. The polynucleotide of claim 31, wherein said signal sequence comprises MDGRLVFLLASLAIVSDA (SEQ ID NO: 12).

34. The polynucleotide of claim 32 comprising the sequence of Syn-134R (SEQ ID NO:4) or Syn2-134R (SEQ ID NO:6).

35. A vector comprising the polynucleotide of claim 26.

36. A cell comprising the polynucleotide of claim 26.

37. The cell of claim 36, wherein said cell is a mammalian cell.

38. A method of producing a substantially pure polypeptide comprising:
(a) culturing the cell of claim 36 under conditions that permit expression of the polypeptide encoded by said polynucleotide; and
(b) purifying said polypeptide, thereby producing a substantially pure polypeptide.

39. The method of claim 38, wherein said cell is a mammalian cell.

40. A pharmaceutical composition comprising:
(a) a polynucleotide of claim 26; and
(b) a pharmaceutically acceptable carrier.

41. (canceled)

42. The method of claim 26, wherein said polynucleotide encodes a polypeptide comprising a sequence at least 90% identical to amino acids 19-156 of TPV134R (SEQ ID NO:7).

43. The method of claim 26, wherein said polynucleotide encodes a polypeptide comprising a sequence identical to 19-156 of TPV134R (SEQ ID NO:7).

44.-50. (canceled)

51. A fusion protein comprising:
(a) a myoxma T7 signal sequence; and
(b) a heterologous sequence at least 90% identical to amino acids 19-156 of TPV134R (SEQ ID NO. 7).

52. The fusion protein of claim 51, wherein said signal sequence comprises MDGRLVFLLASLAIVSDA (SEQ ID NO: 12).

53. A polynucleotide encoding the polypeptide of claim 51.

54. The polypeptide of claim 2, wherein said polypeptide is secreted from a mammalian cell.
